(19) United States
(12) Patent Application Publication
(10) Pub. No.: US 2023/0077709 A1
(43) Pub. Date: Mar. 16, 2023

(54) Chamber Disinfection Devices Using Nebulized Fluid And Plasma

(71) Applicant: Xenex Disinfection Services Inc., San Antonio, TX (US)

(72) Inventor: Gregory Fridman, Philadelphia, PA (US)

(21) Appl. No.: 17/989,822

(22) Filed: Nov. 18, 2022

Related U.S. Application Data
(63) Continuation of application No. PCT/US2021/034924, filed on May 28, 2021.
(60) Provisional application No. 62/704,814, filed on May 29, 2020.

(51) Int. Cl. A61L 2/14; A61L 2/24; A61L 2/22

(52) U.S. Cl. CPC A61L 2/14; A61L 2/24; A61L 2/22; A61L 2202/122; A61L 2202/121; A61L 2202/11; A61L 2202/15; A61L 2202/14

(57) ABSTRACT

Disinfection devices are provided which include an enclosure comprising an interior cavity, a closable loading port for providing access to the interior cavity, a cold plasma generator, a fan, a nebulizer and optionally a heating element to heat the interior cavity. In some cases, the cold plasma generator, the fan, and the nebulizer are arranged in the disinfection device such that plasma species generated by the cold plasma generator are routed to an area to which droplets formed by the nebulizer are discharged and the interior cavity is exposed to a mixture of the droplets and the plasma species. In some additional or alternative cases, the disinfection device includes an electronic control system comprising a processor and a storage medium comprising program instructions executable by the processor for terminating operation of the cold plasma generator after a set time period subsequent to terminating operation of the nebulizer.

### PRIORITY CLAIM

The present application is a continuation application of International Application No. PCT/US2021/034924 filed May 28, 2021, which designates the United States and claims priority to U.S. Provisional Patent Application No. 62/704,814, filed May 29, 2020.

### BACKGROUND OF THE INVENTION

### 1. Field of the Invention

This invention generally relates to disinfection devices using nebulized fluid and plasma and, more particularly, to all-in-one disinfection devices by which to disinfection one or more objects using nebulized fluid and plasma.

### 2. Description of the Related Art

The following descriptions and examples are not admitted to be prior art by virtue of their inclusion within this section.

In general, germicidal systems are designed to subject one or more surfaces and/or objects to a germicide to deactivate or kill microorganisms residing upon the surface/s and/or object/s. A challenge in many applications is sufficiently disinfecting an item without damaging or changing the structure of the item. For instance, harsh chemicals such as bleach, hydrogen peroxide or chlorine are often used to disinfect or sterilize objects, but the aggressive nature of the chemicals can corrode some materials. In addition, boiling water and steam are sometimes used to disinfect or sterilize objects, but exposure to such high temperature fluids can also corrode or cause materials to deform. Another challenge in many germicidal applications is sufficiently accessing all surfaces of an object to insure thorough disinfection of the object. In particular, some germicidal systems may only effectively treat surfaces which are facing the germicidal system and, thus, surfaces not facing the system may not be disinfected adequately. Yet another challenge in many germicidal applications is providing a readily accessible device or system by which to disinfect items relatively quickly, particularly small and/or frequently used items. In particular, many germicidal systems offer large batch processing in order to disinfect several items at the same time, but the process of loading, treating, and unloading the items is time-consuming and the systems themselves are generally bulky and expensive.

Objects which are often in need of frequent and quick disinfection are those used in a setting in which the transmissions of pathogens are high, such as but not limited to a healthcare setting. For example, personal protective equipment (PPE), such as but not limited to masks, gowns, face shields, and goggles, is a necessary element for medical professionals and other individuals who wish to protect themselves from viral and bacterial introduction from a third party. Often, the third party is a patient who is presenting themselves to a medical clinic for treatment and who may be showing symptoms of high viral or bacterial loads, causing infection. Other objects that are often in need of frequent and quick disinfection in a healthcare setting are those which are frequently handled by patients or personnel, such as but not limited to stethoscopes, blood pressure cuffs and television remotes.

Frequent disinfection and PPE have become increasingly important under the current climate of newly introduced coronaviruses. While prior coronavirus infections, including SARS and MERS, produced high mortality rates, their rate of transmission remained low. However, COVID-19, another coronavirus, currently shows much higher transmission rates and a yet-to-be-defined mortality rate. Accordingly, medical personnel must use appropriate protection to prevent the spread of viral loads and the creation of fomites in hospital and outpatient settings. Specifically, the goals remain to prevent transmission to medical professionals, staff, and patients at these medical settings and in public areas.

Some items used in a healthcare setting are intended for single use and, thus, the items are not designed withstand a disinfection process. However, in cases in which the number of patients increases dramatically in a short period of time, such has occurred during the Covid-19 pandemic, the continual replacement of single use items has led to reductions in their availability. As a result, the predicament has forced multiple use of items intended for single use, particularly with regard to PPE, increasing the risk of fomites leading to incidental transmission. Moreover, even in times that reuse of “single use” items is not required, there is a significant amount of waste attributed with the items since they are intended for single use. Furthermore, some single use medical supplies, such as but not limited to alcohol wipes, bandages, and gloves, are stored in a setting (such as a patient room) for ready access and, thus, are susceptible to being exposed to pathogens without even being used. In such cases, exposed unused items may be disposed of since they are often difficult and/or time consuming to disinfect due to the sheer number of them and the lack of readily available disinfection devices suitable to disinfect the items in a quick manner without causing damage to them.

Accordingly, there is a need for disinfection devices that do not cause damage to items being disinfected, sufficiently access all surfaces of items being disinfected in a relatively short amount of time and are economical.

### SUMMARY OF THE INVENTION

Disinfection devices and methods of their use are provided. The following description of various embodiments of the apparatuses and methods is not to be construed in any way as limiting the subject matter of the appended claims.

Embodiments of disinfection devices include an enclosure comprising an interior cavity, a closable loading port for providing access to the interior cavity, a cold plasma generator, a fan, a nebulizer and optionally a heating element to heat the interior cavity. In general, the disinfection device is configured such that plasma species generated by the cold plasma generator and droplets generated by the nebulizer are discharged in the interior cavity. In addition, the fan is arranged in the disinfection device to move the plasma species and the droplets in the disinfection device. In some cases, the cold plasma generator, the fan, and the nebulizer are arranged in the disinfection device such that plasma species generated by the cold plasma generator are routed to an area to which droplets formed by the nebulizer are discharged and the interior cavity is exposed to a mixture of the droplets and the plasma species. In some additional or alternative cases, the disinfection device includes an electronic control system comprising a processor and a storage medium comprising program instructions executable by the processor for activating the fan, the cold plasma generator, and the nebulizer. In some of such cases, the storage medium further includes program instructions for terminating operation of the nebulizer and terminating operation of the cold plasma generator after a set time period subsequent to terminating operation of the nebulizer.

An embodiment of a method includes placing disposable personal protective equipment into a disinfection chamber comprising a cold plasma generator, a fan, a container comprising fluid, a nebulizer, and a closable loading port. The method further includes closing the loading port and activating a disinfection process to be performed in the disinfection chamber subsequent to closing the loading port.

### BRIEF DESCRIPTION OF THE FIGURES

Other objects and advantages of the invention will become apparent upon reading the following detailed description and upon reference to the accompanying drawings in which:

FIG. 1 illustrates a front perspective view of a disinfection device with depicted electronic features and exterior components on the front and side of the device as well as open views to internal components;

FIG. 2 illustrates a cross-sectional view of the disinfection device depicted in FIG. 1 taken along A-A;

FIG. 3 illustrates a schematic view of an alternative arrangement of components for a disinfection device;

FIG. 4 illustrates a schematic view of another alternative arrangement of components for a disinfection device;

FIG. 5 illustrates a schematic view of yet another alternative arrangement of components for a disinfection device;

FIG. 6 illustrates a flowchart of a process for disinfecting one or more objects in a disinfection device comprising a cold plasma generator, a nebulizer, and a fan; and

FIG. 7 illustrates a flowchart of another process for disinfecting one or more objects in a disinfection device comprising a cold plasma generator, a nebulizer, and a fan.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE INVENTION

Disinfection devices disclosed herein generate cold plasma-generated reactive oxygen and nitrogen species (RONS) to disinfect items. Although cold plasma technology has been experimented with to decontaminate produce, such processes have been limited to makeshift systems without consideration of efficiency, costs, ease of use, or longevity of the system components. The disinfection devices disclosed herein, however, include several features and designs taking such factors into consideration, particularly for but not limited to disinfection devices having all components incorporated into single unit. For instance, as explained in more detail below, the disinfection devices disclosed herein are designed to generate droplets having dissolved reactive species without shorting the electrodes of the cold plasma generator despite the limited confines of the device in which the droplets are formed and circulated. In addition, the disinfection devices disclosed herein are designed to enable and emphasize the fissure of water droplets to achieve greater germicidal efficacy in a shorter amount of time. Furthermore, the disinfection devices disclosed herein are designed with components which are more economical and readily available than what was used in previous devices using cold plasma technology to disinfect produce. Moreover, the disinfection devices disclosed herein are designed to reduce the cycle time by which items may be retrieved relative to previous devices using cold plasma technology to disinfect produce.

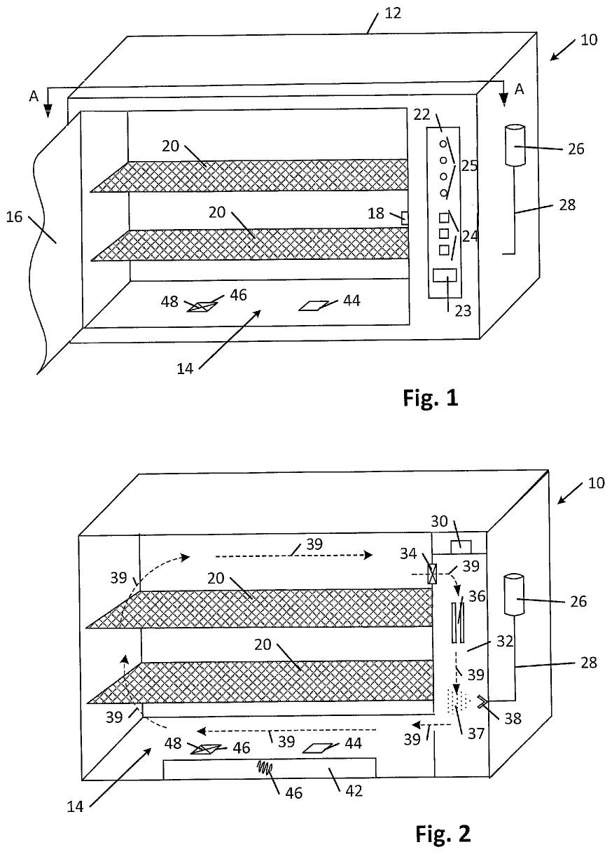

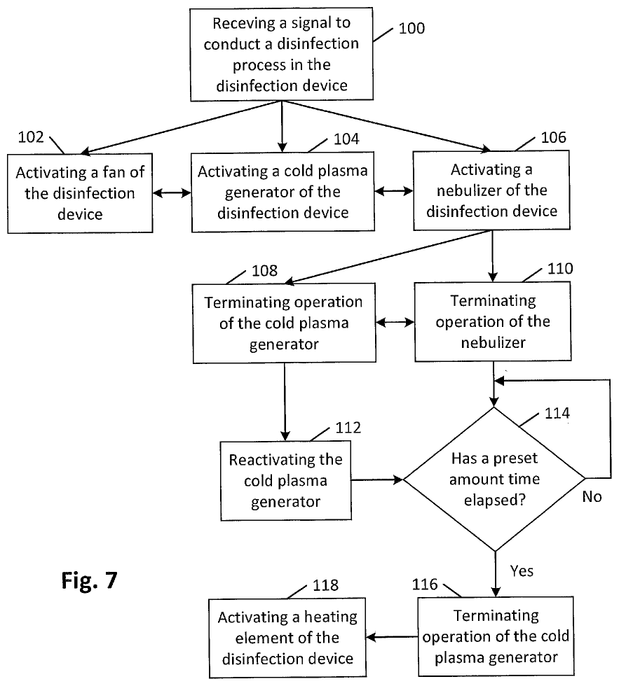

As set forth in more detail below, each of the disinfection devices disclosed herein which include an enclosure comprising an interior cavity, a closable loading port for providing access to the interior cavity, a cold plasma generator, a fan, a nebulizer, a fluid supply, and optionally a heating element to heat the interior cavity. The arrangement of the components may vary as discussed in more detail below in reference to FIGS. 1-5. In addition to such components, the apparatuses may include a programmable controller (i.e., a storage medium comprising a processor and program instructions executable by the processor) for automated operations of the apparatuses. FIG. 7 depicts a flow chart of example processes which may be automated via such program instructions while FIG. 6 illustrates a flow chart of the general methodology used for disinfecting one or more objects in the disinfection devices. As will be set forth in more detail below, the apparatuses and components described herein are not limited to the depictions in the drawings. Several other arrangements of components and/or configurations of apparatuses may be considered. Furthermore, it is noted that the drawings are not necessarily drawn to scale.

As noted above, each of the disinfection devices disclosed herein include a cold plasma generator. The term “cold plasma” as used herein refers to a plasma which is not in thermodynamic equilibrium, particularly that the temperature of the electrons is much higher than the temperature of ions and neutrals. The term “cold plasma” as used herein is synonymous with the terms “non-thermal plasma” and “non-equilibrium plasma”. The cold plasma generators of the disinfection devices disclosed herein may include any generator known to generate a cold plasma. Examples of cold plasma generators which may be used for the disinfection devices disclosed herein include but are not limited to glow discharge, corona discharge, atmospheric pressure plasma jet, dielectric barrier discharge, micro-hollow cathode discharge, plasma needle, and low-pressure plasma. Furthermore, the cold plasma generators considered for the disinfection devices disclosed therein may include pulsed cold plasma generators or continuous wave cold plasma generators.

Both pulsed and continuous-wave dielectric barrier discharge cold plasma generators were used in the development of the disinfection devices disclosed herein and are known to function particularly well with the design considerations discussed herein. Advantages of dielectric barrier discharge cold plasma generators is small size, making them easily configured, and deployed into an enclosure. Continuous wave dielectric barrier discharge cold plasma generators are advantageous due to their availability and lower costs as compared to pulsed dielectric barrier discharge cold plasma generators. Yet, a disadvantage of employing continuous wave dielectric barrier discharge cold plasma generators is that they generate considerably more ozone in a given disinfection process as compared to pulsed dielectric barrier discharge cold plasma generators. The disinfection devices disclosed herein, however, are designed to remove the ozone prior to the end of a disinfection cycle as described in more detail below in reference to FIG. 6.

Each of the disinfection devices disclosed herein also include a nebulizer. While any type of nebulizer may be used in the disinfection devices disclosed herein, it preferable to utilize a nebulizer that is configured to generate droplets having an average diameter of 5 μm or smaller. (The size of the microdroplets follows a bell curve on the size distribution, and thus there is variance in the size of the microdroplets.) In particular, small droplets not only allow for saturation of the interior cavity of the disinfection device in which the items are being disinfected but also increases the rate of transmission of the RONS species into the droplets because of the high surface area of the droplets. Thus, the smaller particle sizes are significantly more effective at reducing viral or bacterial loads as compared to larger fluid droplets.

A further component of the disinfection devices disclosed herein is a fluid supply line coupled to the nebulizer. In some cases, a disinfection device may include a refillable container coupled to the supply line for a user to supply fluid to prior to a disinfection process. Alternatively, a disinfection device may be configured to receive pre-filled containers of fluid at the supply line. In yet other cases, the disinfection device may be configured for attachment to a piped fluid line provided in an area or room in which the disinfection device is to be used. In some cases, a disinfection device may include multiple fluid supply containers and/or multiple fluid lines, each having a supply of a different fluid, such as water and various additional excipients. In such cases, a user may be able to configure a particular fluid mixture for a disinfection process. In addition or alternatively, a user interface of the disinfection device may offer different modes for disinfection, wherein one or more of the disinfection modes are associated with a different fluid make-up and the disinfection device is programmed to supply the appropriate fluid make-up for a selected disinfection mode. The different disinfection modes and, thus, the different fluid make-ups may be dependent on a number of variables, including but not limited to the type of object/s to be disinfected and/or a microorganism/s to be targeted during the disinfection process.

In general, the fluid fed to the nebulizer during a disinfection process includes water and, in some cases, it may further comprise additional excipients such as: acids, bases, buffer solutions (comprising a conjugate acid and base), peroxide solutions, bleach, peracetic acid, etc. The percentage of the additional excipients may vary depending on the application of the disinfection process, particularly the material, configuration, size, shape, or number of items to be disinfected and/or the microorganism/s to be targeted during the disinfection process. A collective concentration of one or more excipients in a fluid fed to the nebulizer may generally vary between 0.01% to approximately 20%. In yet other embodiments, the fluid to be atomized for the disinfection process may consist essentially of water. Thus, the amount of water in the fluid supply for a disinfection process may vary between approximately 80% and 100%. The water in the fluid may be tap water, deionized water, reverse osmosis deionized water, or purified water. For some disinfection processes, tap water may be advantageous since it contains minerals and dissolved solids including metallic ions which are useful in protecting the reactive species generated in plasma.

Turning to the drawings, FIG. 1 shows disinfection device 10 having enclosure 12 with interior cavity 14 and door 16 for providing access to the interior cavity (door 16 is shown as a partial portion in FIG. 1). The door allows for selective opening and closing of the device, forming a closable loading port for the device. Door 16 is configured to create an airtight seal under room pressure when closed. The disinfection devices disclosed herein are not pressurized or depressurized, so room pressure is simply the ambient pressure at the location of the device and the seal to maintain an airtight fit is minimal as compared to those chambers that may be pressurized. Furthermore, door 16 may be a hinged door, a sliding door, or a retractable door and, thus, the disinfection devices disclosed herein should not be limited to having a hinged door. Regardless of the type of door employed, disinfection device 10 may include sensor 18 along the frame of the loading port into interior cavity 14 to detect whether the door is open or closed. In addition, disinfection device 10 may include a locking device to secure door 16 in a closed position for a disinfection cycle. In any case, the size of disinfection device 10 and, more particular, the size of interior cavity may depend on the size of the objects intended to be disinfected in the device. An example volume for interior cavity 14 may be between approximately 0.1 liters and approximately 100 liters, making it a device that can be placed in a variety of environments and, thus, readily available.

As shown in FIG. 1, disinfection device 10 may include user interface 22 having input controls 24 to affect operation of disinfection device 10, such as but not limited to a start and stop button 23 to enable a user to start and terminate an operation of disinfection device 10 and/or input controls allowing selection of different operation modes conducted by the apparatuses. Configurations of input controls may include any of those known in the art, including but not limited to buttons, switches, graphical user interfaces, touch sensor means and means for enabling audible input. In some cases, user interface 22 may include information conveying devices, such as indicator lights 25 shown in FIG. 1, to inform a user of the apparatus about the status or operation of disinfection device 10. Examples of information regarding the status or operation of disinfection device 10 which may be conveyed on user interface 22 include but are not limited to the status of the door lock, indication if a disinfection cycle is in process or complete, the phase of the disinfection cycle (e.g., formation of plasma induced droplets, dwell time, additional plasma generator or dry cycle as explained in more detail below), duration of the disinfection cycle or the time remaining, and/or errors in the operation of disinfection device 10. In any case, the information conveying device/s may include any visual indicator, visual display or audible means known in the art and, thus, the disinfection devices should not be limited to indicator lights shown in FIG. 1.

In order for user interface 22 to affect the operation of disinfection device 10 and further convey information regarding its status and operation, disinfection device 10 includes a programmable microcontroller device, such as but not limited to an Arduino controller. In other words, disinfection device 10 includes a storage medium having processor and program instructions executable by the processor to affect operations of the device. More specifically, the microcontroller is in electrical communication (wired or wireless) with components of disinfection device 10 to affect and control their operation. For example, the controller may include program instructions to turn on and off certain components of disinfection device 10 in a particular sequence. More specifically, the controller is connected electronically to fluid supply 26, heating element 40, nebulizer 38, dielectric barrier discharge 36, and fan 34. Examples of program instructions for affecting operation of the disinfection devices disclosed herein are described in more detail in reference to FIG. 7.

The term “program instructions”, as used herein, refers to commands within software which are configured to perform a particular function, such as but not limited to one or more of the processes described in reference to FIG. 7. It is noted that the disinfection devices disclosed herein may include program instructions for performing processes other than those specifically described herein and, thus, the disinfection devices are not limited to having program instructions for performing the operations described in reference to FIG. 7. In any case, program instructions may be implemented in any of various ways, including procedure-based techniques, component-based techniques, and/or object-oriented techniques, among others. In addition, program instructions may be transmitted over or on a carrier medium such as a wire, cable, or wireless transmission link. In general, program instructions may be stored in a storage medium within the devices described herein. The term “storage medium”, as used herein, refers to any electronic medium configured to hold one or more set of program instructions, such as but not limited to a read-only memory, a read-write memory, a random-access memory, a magnetic or optical disk, or magnetic tape.

As shown in FIG. 1, at least one support structure 20 is positioned within interior cavity 14 to hold one or more objects. Support structure 20 may be a shelf, tray, or basket and, in cases in which a disinfection device includes multiple support structures, the support structures may include any combination of one or shelves, one or more trays, one or more baskets and/or one or more hooks. Regardless of the type of support structures arranged in interior cavity 14, the one or more support structures may include openings to allow for the flow of plasma induced droplets through the support structures, particularly such that underside surfaces of one or more objects arranged on the one or more support structures may be exposed to the droplets. In some embodiments, one or more of support structures 20 may be readily removeable from interior cavity and, in some cases, repositionable within interior cavity 14. In other cases, one or more of support structures 20 may be fixedly arranged within interior cavity 14 such that they are not readily removable. In any case, the configuration (e.g., the shape, size, type, and position in the interior cavity) of structures 20 may vary and, thus, the disinfection devices disclosed herein should not be limited to the depiction in FIG. 1. For example, in some cases, a disinfection device may additionally or alternatively include one or more hooks attached to the ceiling and/or sidewalls of interior cavity 14 to hang objects therein. Although support structures are advantageous for allowing a greater number of objects to be placed in the disinfection device for a given disinfection process and further expose underside surfaces of the objects to plasma induced droplets when the support structure include openings, disinfection device 10 may, in some cases, be void of a support structure to hold an object.

Disinfection device 10 further includes fluid supply container 18 arranged along an exterior side surface of the device. Such a position or arrangement along any other exterior surface of disinfection device 10 permits ready access to the container regardless of whether door 16 is open or closed. In alternative embodiments, fluid supply container 26 may be disposed inside disinfection device 10. In some of such cases, fluid supply container 26 may be disposed in interior cavity 14 such that a user has access to the container upon opening door 16. In yet other embodiments, fluid supply container 26 may be disposed in a chamber of disinfection device 10 separate from interior cavity 14. In such cases, disinfection device 10 may include a supply port along its exterior or within interior cavity 14 that allows a user to supply fluid to the container. In addition or alternatively, disinfection device 10 may include a separate door (i.e., distinct from door 16) along its exterior surface, which provides access to the fluid supply container, particularly to supply fluid thereto and/or to remove the container. In any case, disinfection device 10 includes a fluid line extending from the fluid supply 26 and terminates at a nebulizer within the device, such as shown by fluid line 28 in FIG. 1.

Turning to FIG. 2, a cross-sectional view of disinfection device 10 taken along A-A of FIG. 1 is illustrated, showing the arrangement of other components of the disinfection device arranged in chambers separate from interior cavity 14. FIG. 2 further depicts an example air flow pattern through the disinfection device in accordance with the operation of some of the components as explained in more detail below. As noted above, disinfection device 10 may include a microcontroller for controlling the operation of components of the device. FIG. 2 illustrates microcontroller 30 within the confines of disinfection apparatus above chamber 32 in which fan 34, dielectric barrier discharge 36 and nebulizer 38 is arranged. Microcontroller 30, chamber 32, fan 34, dielectric barrier discharge 36, and nebulizer 38, however, may be arranged in other positions in disinfection device 10 and, thus, the disinfection devices disclosed herein should not be limited to the arrangement of components depicted in FIG. 2.

As shown in FIG. 2, chamber 32 includes a first opening 31 and a second opening 33 for providing airflow passage between the chamber and interior cavity 14. Fan 34 is positioned within first opening 31 to force (i.e., push or pull) air into chamber 32, making first opening 31 serve as an air inlet for the chamber. Fan 34 may alternatively be positioned exterior or interior to first opening 31 to serve the same purpose. In any case, air is routed through chamber 30 to second opening 33, which serves as an air outlet for the chamber. In route, the air passes over dielectric barrier discharge 36 which is charged at a high voltage to generate plasma. As noted above, other types of cold plasma generators may be used for the disinfection devices disclosed herein and, thus, dielectric barrier discharge 36 may be substituted with a different cold plasma generator in disinfection device 10.

Because air contains oxygen, nitrogen, and hydrogen, reactive oxygen and nitrogen species (RONS), such as but not limited to ozone (O₃), hydroxyl radical (OH), hydrogen peroxide (H₂O₂), singlet oxygen (O₂*), and/or peroxynitrite radical (ONOO*) are generated in the plasma. The RONS, most specifically the ozone, peroxides, and peroxynitrite, are strong oxidizing species and, thus, provide dramatic disinfection, particularly log reductions of pathogens, including bacteria, viruses, and spores. However, RONS are typically unstable. Accordingly, there is a need to stabilize the reactive species so that they can then attack pathogenic materials. In the disinfection devices disclosed herein, RONS are stabilized by dissolving them into droplets discharged from nebulizer. To affect such a mixture in disinfection device 10, nebulizer 38 is positioned downstream from dielectric barrier discharge 36 to accept and atomize fluid from the fluid line 28 as shown in FIG. 2. The RONS generated by dielectric barrier discharge 36 are admixed with droplets formed by nebulizer 38, allowing at least some of the RONS to be dissolved into the droplets. The fluid of the droplets protects the reactive species from being destroyed and increases their lifetime.

As set forth above, when fluid is expressed from a nebulizer of a disinfection device disclosed herein, it creates very small and fine droplets, specifically having an average diameter of 5 μm or smaller. The size of the droplets follows a bell curve of size distribution and, thus, there is variance in the size of the droplets. The small size of these droplets increases the rate of transmission of the RONS species into the microdroplets because of the high amount of surface area among the many droplets relative to if larger droplets are formed. Furthermore, once generated, the greater surface area and sheer number of droplets ensures that the droplets having dissolved RONS are in sufficient quantities and density to saturate the surface of the objects being disinfected in interior cavity 14.

As shown in FIG. 2, the mixture of droplets and RONS, whether or not dissolved into the droplets, are expressed out of second opening 33 into interior cavity 14. In some cases, the flow rate of fan 34 (and/or the inclusion of one or other fans in disinfection device 10) may be such that the charged particles and droplets are circulated through nearly the entirety of interior cavity 14, such as but not limited to the flow path indicated by arrows 39 in FIG. 2. In order to affect dispersal of the charged particles and microdroplets throughout interior cavity 14, the flow rate will depend on the volume of interior cavity. An example range of flow rates for a disinfection device having an interior cavity volume between approximately 0.1 liter and approximately 100 liters may be between approximately 0.1 m³/min and approximately 0.5 m³/min and, in some cases, between approximately 0.25 m³/min and approximately 0.35 m³/min.

In some cases, the arrangements of fan 34 and/or the arrangement of one or other fans in disinfection device 10 may be to affect a particular flow path pattern in interior cavity 14, such as but not limited to the flow path indicated by arrows 39 in FIG. 2. In particular, it has shown to be advantageous to route some of the droplets discharged into interior cavity 14 (whether or not they have dissolved RONS) through dielectric barrier discharge 36 (or any other type of cold plasma generator) to cause fissure of the droplets, particularly to obtain droplets having sub-micron diameters. Such a process further advantageously increases the available surface area for the RONS to dissolve into, which increases the germicidal efficacy of the disinfection process. It is important, however, to not short the electrodes of the cold plasma generator. It has been found that droplets having a diameter of less than 10 μm can travel through a dielectric barrier discharge cold plasma generator without shorting its electrodes, particularly since the viscosity of air can oppose gravity for droplets of this size. In other words, droplets having a diameter of less than 10 μm can generally stay suspended in air (such as a mist) and, thus, can move freely with air through dielectric barrier discharge 36 without depositing on its electrodes. Such a phenomenon, however, dissipates with droplets of greater diameters.

In order to prevent droplets having a diameter of greater than 10 μm from entering the cold plasma generators of the disinfection devices disclosed herein, the disinfection devices are designed to route droplets generated from their nebulizers along a path and at a speed through their interior cavities which provides time for droplets having a diameter of greater than 10 μm to fall out of suspension in the air flow by gravity and/or to smash into the interior walls, ceiling and/or flooring before entering the cold plasma generator. Part of such a design is the arrangement of one or more fans in the disinfection devices to affect a particular flow path pattern and air flow speed in their interior cavities. Another part of such a design is the relative placement of the nebulizer and the cold plasma generator. In particular, it is advantageous to enable a flow path of approximately 1 meter or more between the output of the nebulizer and the inlet of the cold plasma generator. In general, the arrangement of the one or more fans, nebulizer, and cold plasma generator in a disinfection device to affect such an objective will depend on the size of its interior cavity.

In some cases, it may be advantageous to arrange the nebulizer and the cold plasma generator along the same sidewall of the disinfection device or both along the ceiling or flooring of the disinfection device, or even in relation to the same corner of the disinfection device, to maximize the potential length of a flow path through the interior cavity of the disinfection device, such as generally shown by arrows 39 in FIG. 2. The route of flow paths in the interior cavities of the disinfection devices disclosed herein, however, are not limited to being circular or elliptical. In particular, one or more fans may be arranged to form a flow path that traverses the length and/or depth of the interior cavity multiple times prior to entering into the cold plasma generator. Other flow patterns may be considered as well.

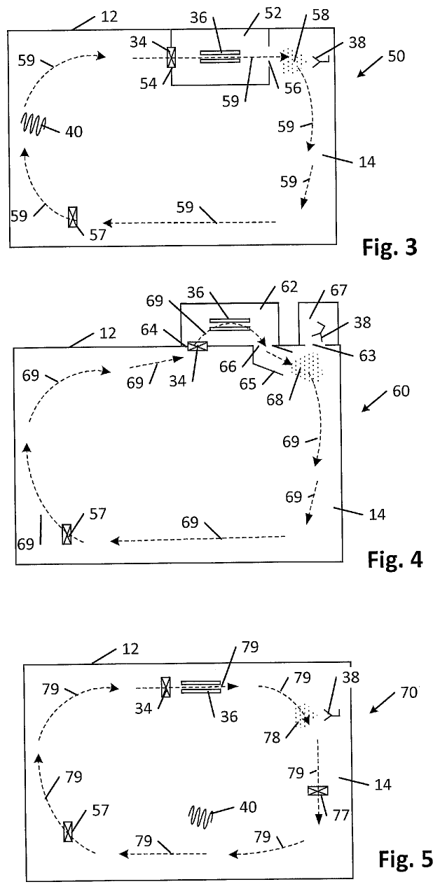

In some cases, the cold plasma generator, the fan, and the nebulizer may be arranged in a disinfection device such that plasma species generated by the cold plasma generator are routed to an area to which droplets formed by the nebulizer are discharged. Disinfection device 10 depicted in FIG. 2 depicts an example of such an arrangement. In particular, FIG. 2 illustrates nebulizer 38 arranged in chamber 32 downstream from dielectric barrier discharge 36 such that plasma species generated by dielectric barrier discharge 36 are routed to area 37 to which droplets formed by the nebulizer are discharged. As noted above, fan 34 is arranged within first opening 31 to force (i.e., push or pull) air into chamber 32, making first opening 31 serve as an air inlet for the chamber. Fan 34 may alternatively be positioned exterior or interior to first opening 31 to serve the same purpose. Examples of other arrangements of a cold plasma generator, a fan, and a nebulizer in a disinfection device which facilitate plasma species generated by the cold plasma generator being routed to an area to which droplets formed by the nebulizer are discharged are illustrated in FIGS. 3-5 and described in more detail below.

As noted above, the disinfection devices disclosed herein may, in some cases, include a heating element to heat the interior cavity, particularly to dry objects in the interior cavity which have been exposed to the droplets discharge from the nebulizer and further to destroy ozone generated by the cold plasma generator during the disinfection process. Disinfection device 10 in FIG. 2 illustrates heating element 40 in chamber 42) separate from interior cavity 14 and chamber 32. However, heating element 40 may alternatively be arranged in interior cavity 14 and chamber 32. In such cases, chamber 42 may be omitted from the disinfection device, but in other cases, it may be used to circulate the flow of air, droplets, RONS and/or heat within interior cavity without having heating element 40 therein. In any case, chamber 42 includes two openings 44 and 46, the latter of which has fan 48. Fan 48 pulls or pushes air into or out of heating chamber 42 to circulates air therethrough to opening 44. When heating element 40 is operated, air passing over and across it is heated and passes through opening 46 to heat interior cavity 14.

As described in more detail below, in embodiments in which a disinfection device disclosed herein includes a heating element, the disinfection device may be configured to heat its interior cavity to a temperature between approximately 40° C. and approximately 60° C., in some embodiments, to a temperature of approximately 50° C. In some cases, a disinfection device disclosed herein may include a temperature sensor in its interior cavity, which sends signals to the microcontroller to control the temperature of the heating element to ensure a 50° C.±2° C. temperature is reached and maintained in the interior cavity during a heating cycle. In other cases, the heating element may be a coil style heater applying a known load, which is calibrated based on the airflow and space to reach the noted temperature ranges. As such, in such cases, an on/off cycle of the heating element may not be needed to reach and maintain the noted temperature ranges. However, to the extent that control is desired, the temperature probe can be utilized by the microcontroller to activate or deactivate the heating element or the fan to modify the temperature.

Given the aforementioned description of disinfection device 10 in FIG. 2, it is noted that a cold plasma generating device for disinfecting one or more objects is provided herein which include: a three chamber disinfecting device, comprising a cleaning chamber, a plasma chamber, and a heating chamber; a first and second room partition configured to: (i) separate the cleaning chamber from the plasma chamber and (ii) separate the cleaning chamber from the heating chamber; wherein each of the first and second room partitions comprise a fan at a first end and an opening at a second end, wherein activation of the fan exchanges air between the cleaning chamber and the plasma or heating chambers; an electronic control system including at least one computer processor configured to execute computer readable instructions to perform a disinfection protocol; and wherein the at least one computer processor is configured to selectively control a plasma generating dielectric barrier discharge located within the plasma chamber, a heating element located in the heating chamber, and a fan positioned on each of the first and second room partitions according to the disinfection protocol.

As noted above, FIGS. 3-5 illustrate schematic drawings of disinfection devices having a cold plasma generator, a fan, and a nebulizer arranged such that plasma species generated by the cold plasma generator are routed to an area to which droplets formed by the nebulizer are discharged. In particular, FIGS. 3-5 illustrate schematic drawings of disinfection devices 50, 60, and 70 having different arrangements of a cold plasma generator, a fan, and a nebulizer for such an objective. It is noted that other arrangements of such components may be used to achieve such an objective and, thus, the disinfection devices disclosed herein should not be limited to the examples of component arrangements described in reference to FIGS. 2-5. Furthermore, it is noted that disinfection devices 50, 60, and 70 may include any of the components described in reference to disinfection device 10 of FIG. 2. Some of the features of disinfection device 10 of FIGS. 1 and 2 have been omitted from the illustrations of disinfection devices 50, 60 and 70 (e.g., door 16, sensor 18, support structures 20, user interface 22 and all of its input controls and information conveying devices, fluid container 26 and fluid line 28 and microcontroller 30) to simplify the drawings of FIGS. 3-5. Furthermore, the features depicted in FIGS. 3-5 with the same reference numbers as shown in FIGS. 1 and 2 (i.e., enclosure 12, interior cavity 14, fan 34, dielectric barrier discharge 36, nebulizer 38 and heating elements 40) include the same configurations as described in reference to FIGS. 1 and 2. Descriptions of such features are not reiterated for the sake of brevity.

As shown in FIG. 3, disinfection device 50 includes chamber 52 distinct from interior cavity 14, which has opening 54 and opening 56 for providing air flow between the chamber and interior cavityl4. Fan 34 is positioned within opening 54 to force (i.e., push or pull) air into chamber 52, making opening 54 serve as an air inlet for the chamber. Fan 34 may alternatively be positioned exterior or interior to opening 54 to serve the same purpose. In any case, air is routed through chamber 52 to opening 56, which serves as an air outlet for the chamber. In route, the air passes over dielectric barrier discharge 36 which is disposed in chamber 52. Nebulizer 38 is arranged in interior cavity 14 such that plasma species generated by dielectric barrier discharge 36 are routed to area 58 to which droplets formed by the nebulizer are discharged as shown in FIG. 3. Alternatively stated, nebulizer 38 is arranged in interior cavity 14 to discharge droplets toward an area in the interior cavity adjacent opening 56. The placement of chamber 52 in disinfection device 50 differs from the placement of chamber 30 in disinfection device 10 to emphasize that a chamber of the disinfection devices disclosed herein may be arranged along any interior surface of the disinfection device, including any of the sidewalls, ceiling, flooring or even an interior surface of the door for accessing interior cavity 14 of the disinfection device. The placement of chambers 30 and 52 in disinfection devices 10 and 50 are not mutually exclusive to their arrangement of components and, thus, chambers 30 and 52 and the components disposed therein and/or in proximity to their openings may be arranged along any interior surface of the disinfection device. Chambers suspended from such interior surfaces may also be considered for the disinfection devices considered herein.

Disinfection device 50 further differs from disinfection device 10 with nebulizer 38 arranged in interior cavity 14 and being arranged along a different sidewall surface than chamber 52. Such features are not mutually exclusive. In particular, chamber 52 could be extended to encase nebulizer 38 with opening 56 moved to the bottom of the chamber such that a mixture of RONS and droplets could be discharged into interior cavity 14. In yet other cases, nebulizer 14 could be alternatively arranged along the ceiling of disinfection device 50 discharging droplets downward to area 58. Such an arrangement may be advantageous for aligning the flow of droplets with airflow pattern indicated by arrows 59. In particular, discharging the droplets downward rather than toward opening 56 of chamber 52 may aid in preventing droplets larger than 10 μm from entering the chamber and potentially shorting dielectric barrier discharge 36. In other cases, however, the airflow through chamber 52 as governed by fan 34 may be sufficient to prevent such large droplets from entering opening 56 and, thus, the direction of discharge from nebulizer may not be of concern.

In any case, nebulizer 38 is spaced a sufficient distance from opening 56 to prevent such large droplets from entering the opening. However, such an objective should be balanced with the objective to route the RONS discharged from dielectric barrier discharge 36 to the area at which a high concentration of droplets from nebulizer 38 are discharged (depicted as area 58 in FIG. 3). In particular, the distance between opening 56 and nebulizer 38 should be balanced to optimize the concentration of RONS routed to an area having a high concentration of droplets to facilitate the dissolving of RONS into the droplets but also to prevent droplets larger than 10 μm from entering opening 56. In some cases, nebulizer 38 may be arranged approximately 12 inches or less from opening 56 and, more specifically, approximately 6 inches or less from opening 56.

As further shown in FIG. 3, disinfection device 50 may include fan 57 arranged to facilitate, along with fan 34, the airflow pattern depicted by arrows 59. Disinfection device 50 may include any number of one or more additional fans to affect such an airflow pattern as well. It is noted, however, that the inclusion of additional fan 57 in disinfection device 50 is not mutually exclusive to the arrangement of dielectric barrier discharge 36 and nebulizer 38 in disinfection device 50. In particular, as noted above, the disinfection devices disclosed herein may include any number of one or more fans to affect an airflow pattern through the disinfection device. FIG. 3 further shows disinfection device 50 having heating element 40 in interior cavity 14 to emphasize that heating elements of the disinfection devices disclosed herein need not be placed in a chamber separate from their interior cavities. It is noted that having heating element 40 in interior cavity 14 is not mutually exclusive to the arrangement of any of the other components in disinfection device 50.

Turning to FIG. 4, disinfection device 60 is shown having chamber 62 distinct from interior cavity 14, which has opening 64 and opening 66 for providing air flow between the chamber and interior cavity 14. Fan 34 is positioned within opening 64 to force (i.e., push or pull) air into chamber 62, making opening 64 serve as an air inlet for the chamber. Fan 34 may alternatively be positioned exterior or interior to opening 64 to serve the same purpose. In any case, air is routed through chamber 62 to opening 66, which serves as an air outlet for the chamber. In route, the air passes over dielectric barrier discharge 36 which is disposed in chamber 62. Nebulizer 38 is arranged in chamber 67, which is distinct from interior cavity 14 and chamber 62. More specifically, nebulizer 38 is arranged in chamber 67 to discharge droplets through opening 63. Chamber 67 includes opening 63 to serve as a fluid outlet for passing droplets formed by nebulizer 38 into the interior cavity 14.

Disinfection device 60 further includes channel 65 extending from opening 66 of chamber 62. As shown, channel 65 is configured such that plasma species generated by dielectric barrier discharge 36 and discharged from opening 66 are routed to area 68 to which droplets formed by the nebulizer are discharged. In this manner, channel 65 and opening 63 are configured to direct their discharge to common area in interior cavity 14. The arrangement of channel 65 and chamber 67 may generally be such that the outlet of channel 65 and opening 63 of chamber 67 are in relatively close proximity, particularly to optimize the concentration of RONS routed to an area having a high concentration of droplets to facilitate the dissolving of RONS into the droplets. For example, the outlet of channel 65 and opening 63 of chamber 67 may be within approximately 12 inches of each other and, more specifically, within approximately 6 inches of each other.

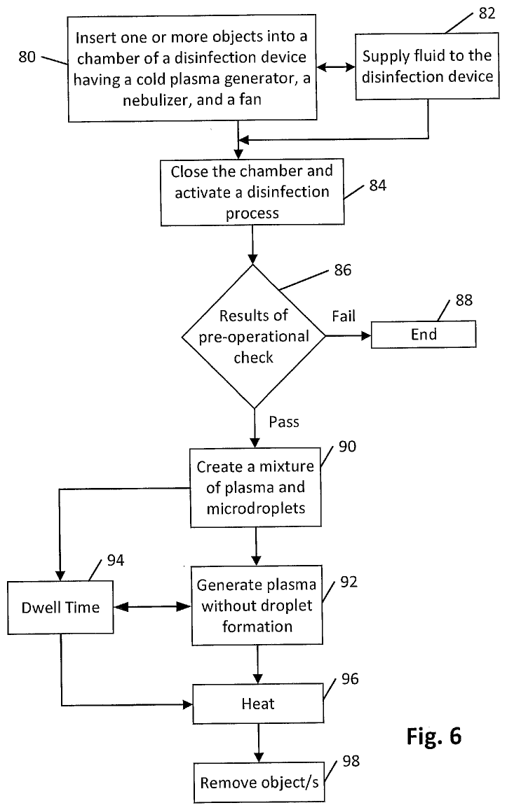

Disinfection device 60 differs from disinfection devices 10 and 30 in that it includes separate chambers to house dielectric barrier discharge 36 and nebulizer 38 and the chambers are arranged exterior to enclosure 12. Such features are not mutually exclusive. In particular, the depiction of disinfection device 60 in FIG. 4 is to emphasize the disinfection devices disclosed herein may include any number of one or more chambers interior to enclosure 12 separate from interior cavity 14 and/or any number of one or more chambers exterior to enclosure 12 separate from interior cavity 14. In any of such cases, the chambers are not limited to housing dielectric barrier discharge 36 and/or nebulizer 38. For example, a chamber of a disinfection device disclosed herein may alternatively house a heating element, such as depicted for disinfection device 10 in FIG. 2. In yet other embodiments, such as depicted for disinfection device 70 in FIG. 5, a disinfection device need not include a chamber separate from interior cavity 14. In any case, in embodiments in which a disinfection device disclosed herein includes multiple chambers exterior to its enclosures, the chambers need not be disposed along the upper surface of the disinfection device. In particular, one or more of the chambers may be alternatively disposed along an exterior sidewall or the back of the disinfection device.

Disinfection device 60 further differs from disinfection devices 10 and 30 by the inclusion of a channel to route plasma species generated by dielectric barrier discharge 36 to an area at which droplets formed by the nebulizer are discharged. Such an element is not mutually exclusive to scenarios in which dielectric barrier discharge 36 and nebulizer 38 are housed in different chambers of the disinfection device. In particular, any of the disinfection devices disclosed herein may include a channel arranged in proximity to an outlet of a cold plasma generator to route plasma species to an area at which droplets formed by a nebulizer are discharged. Another distinction of disinfection device 60 from disinfection devices 10 and 30 is the omission of a heating element. As noted above, a heating element is an optional feature for the disinfection devices disclosed herein. Although it may be advantageous to utilize a heating element to dry objects in interior cavity as well as destroy ozone and other RONS in interior cavity 14 after a disinfection process is complete, such a process is not mandatory as ozone and other RONS will innately decompose on their own in time and the amount of residual moisture in the disinfection device after a disinfection process is complete is minimal. In any case, similar to disinfection device 50, disinfection device 60 may include fan 57 arranged to facilitate, along with fan 34, the airflow pattern depicted by arrows 69 in FIG. 4. Disinfection device 60 may include any number of one or more additional fans to affect such an airflow pattern as well.

As noted above, FIG. 5 illustrates disinfection device 70 not having a chamber separate from interior cavity 14 and, thus, each of the components of the disinfection device are arranged in interior cavity 14. In particular, disinfection device 70 is shown in FIG. 5 having fan 34, dielectric barrier discharge 36, and nebulizer 38 arranged in interior cavity 14. Although disposing dielectric barrier discharge 36 in a chamber offers a confined environment in which to generate and contain a plasma and further route it toward a discharge of a nebulizer, such as described for the disinfection devices illustrated in FIGS. 2-4, such confinement of dielectric barrier discharge 36 is not required to achieve adequate disinfection results using the cold plasma technology disclosed herein. In particular, as long as plasma and droplets are discharged and moved within interior cavity 14, they will mix with each other and cause at least some of the RONS to dissolve into the droplets. To increase the admixture of the RONS and droplets, it is advantageous to arrange fan 34, dielectric barrier discharge 36, and nebulizer 38 such that plasma species are routed to area 78 to which droplets formed by the nebulizer are discharged. Such an objective may be achieved by arranging fan 34, dielectric barrier discharge 36, and nebulizer 38 and optionally additional fans, such as fans 57 and 77 as shown in disinfection device 70, to facilitate an airflow pattern which enables such a routing of the plasma species, such as but not limited to the air flow pattern indicated by arrows 79 in FIG. 5. In addition, arranging dielectric barrier discharge 36 such that its output is within approximately 12 inches, or more specifically within approximately 6 inches of the nozzle of nebulizer 38 may further aid in such an objective.

It is noted that the disinfection devices disclosed herein are not limited to having nebulizer 38 disposed in interior cavity 14 when dielectric barrier discharge 36 is arranged in interior cavity 14. In particular, the disinfection devices disclosed herein may have nebulizer disposed in a chamber separate from interior cavity with dielectric barrier discharge 36 arranged in interior cavity 14.

Turning to FIG. 6, a flow chart is illustrated showing the general methodology used for disinfecting one or more objects in the disinfection devices disclosed herein. A significant benefit of this disinfection protocol is that the device does not have to be specially placed within a working environment due to introduction of harsh chemicals or reactive species. In particular, RONS chemistries yielding nontoxic byproducts when they decompose and, thus, there is no exposure to harsh chemicals during or after the disinfection process. As shown in block 80, one or more objects are inserted into an interior cavity of a disinfection device. Access to the interior cavity is provided through a closable loading port of the disinfection device. In some cases, the process may include placing one or more objects on or in support structure in the interior cavity and/or suspending one or more objects from support structures, such as but not limited to hooks, in the interior cavity. In addition or alternatively, one or more objects may be placed on the flooring of the interior flooring. In general, the support structures are configured to allow for objects to be placed and separated from each other while also allowing sufficient airflow around each object to allow for saturation of the RONS microdroplets and consistent coverage of all surfaces of the objects.

The one or more objects may include any objects in need of disinfection, and which can fit in the interior cavity of the disinfection device, which will vary among units. Objects which may be disinfected by the disinfection devices disclosed herein include but are not limited to personal protective equipment (PPE), such as but not limited to masks, gowns, face shields, and goggles, including PPE designed for single use and those design for multiple uses. Other objects which may be considered and are often found in a healthcare setting are stethoscopes, blood pressure cuffs, television remotes, and medical supplies intended for single use or multiple uses, such as but not limited to alcohol wipes, bandages, and gloves. Several other objects may be considered and are not limited to objects typically found in a healthcare setting. Other settings which often include objects in need of disinfection include personal care environments, clean rooms, food manufacturing and/or processing plants, food handling establishments, pharmaceutical laboratories and plants, childcare facilities, animal care centers, and agricultural buildings.

In any case, the method for disinfecting the objects include supplying fluid to the disinfection as denoted in block 82 in FIG. 6. Such a process may be performed prior to, at the same time, or subsequent to inserting one or more objects into the disinfection chamber as is denoted by the double arrowed line between blocks 80 and 82 in FIG. 6. As noted above, supplying fluid to the disinfection device may be done manually or it may be automated. In either case, supplying fluid to the disinfection device may include filling or replacing a container in the interior or along an exterior of the disinfection device or it may include opening a valve from a supply piped to the disinfection device. As noted above, the fluid includes water and, in some cases, it may further comprise additional excipients such as: acids, bases, buffer solutions (comprising a conjugate acid and base), peroxide solutions, bleach, peracetic acid, etc. The percentage of the additional excipients may vary depending on the application of the disinfection process, particularly the material, configuration, size, shape, or number of items to be disinfected and/or the target microorganism/s to be targeted during the disinfection process. A collective concentration of one or more excipients in a fluid fed to the nebulizer may generally vary between 0.01% to approximately 20%. In yet other embodiments, the fluid to be atomized for the disinfection process may consist essentially of water. Thus, the amount of water in the fluid supply for a disinfection process may vary between approximately 80% and 100%. The water in the fluid may be tap water, deionized water, reverse osmosis deionized water, or purified water. For some disinfection processes, tap water may be advantageous since it contains minerals and dissolved solids including metallic ions which are useful in protecting the reactive species generated in plasma.

As shown in block 84, the interior cavity is closed and a disinfection process is activated subsequent to inserting one or more objects into the interior cavity of the disinfection device. Once a disinfection process is activated, the door is locked and the disinfection device is activated to perform a check on the electronic components of the device. The system uses several different sensors to confirm proper operation of the multiple components in the disinfection device before beginning a disinfection cycle, including but not limited to checking the one or more fans in the unit, confirming operation of the cold plasma generator, and confirming sufficient fluid level in addition to the door check. The fans can utilize sensors on the fans themselves or the electrical resistance to confirm their operation. The operation of the cold plasma generator may utilize a sensor to validate the presence of ozone or plasma, thus indicating operation. For example, an inline ozone sensor, or a UV sensor (to confirm light emission) can be utilized to confirm operation. The microcontroller of the disinfection device, being electrically connected to each of the devices or sensors, performs the appropriate check and indicates success or failure of the elements on the control panel via the information conveying devices. As shown in block 86 of FIG. 6, if the disinfection device passes the pre-operational check, the disinfection cycle starts as indicated by the arrow to block 90. However, if the disinfection device fails the pre-operational check, the disinfection device aborts the disinfection process as shown in block 88 of FIG. 6.

Turning to block 90 of FIG. 6, the disinfection cycle is started by creating a mixture of plasma and microdroplets. Such a process entails generating the droplets via nebulizer and at the same time or nearly the same time activating a fan in the disinfection device to push or pull air across or into a cold plasma generator and applying a sufficient voltage to the cold plasma generator to create a plasma. As set forth in detail above, the disinfection devices disclosed herein are configured to expose its interior cavity to a mixture of droplets and plasma RONS. After a set amount of time, such as but not limited to five minutes, the nebulizer ceases but at least one fan and the cold plasma generator remains on to continue to generate plasma without droplet formation as denoted in block 92 in FIG. 6. Such a process continues for a predetermined duration, such as but not limited to 10 minutes. The total time of the microdroplet and plasma mixture generating phase can be modified as needed to increase or decrease the time to optimize the kill rate.

As shown in block 94 of FIG. 6, the disinfection process may include an optional dwell time subsequent to either of the processes denoted in blocks 90 and 92 and sometimes before the plasma generation cycle denoted in block 92. A dwell time refers to when the cold plasma generator and the nebulizer are turned off, while one or more of fans in the disinfection device remain activated to continue a flow of air in and around the one or more objects being disinfected. As shown in block 96 of FIG. 6, an optional heating cycle may be performed subsequent to either the plasma generation cycle denoted in block 92 or the dwell time cycle denoted in block 94. During the heating cycle, a heating element in the disinfection device is activated, particularly to raise the temperature within the interior cavity to a range approximately 40° C. to approximately 60° C. and in some cases, to about 50 +/−2° C. The heating cycle lasts for a predetermined amount of time, such as but not limited to approximately 10 minutes. In any case, after the optional heat cycle denoted in block 98, after the optional dwell time cycle denoted in block 94, or after the process of plasma generation without droplet formation denoted in block 92, the process may end with removing the one or more objects from the disinfection device.

The temperature range specified above for heating cycle denoted in block 96 is utilized to destroy the residual chemistries including the ozone and other reactive species within the interior cavity of the disinfection device. In particular, the heat cycle destroys the reactive species so that when the door is opened, reactive species are not introduced into the ambient air. Thus, there is no need for a BSL (biosafety level)-2 or -3 condition for operation of the device. The heating cycle may be particularly beneficial if a continuous-wave dielectric barrier discharge is employed in the disinfection device. In particular, such a cold plasma generator can generate in excess of 7,000 ppm ozone in the interior chamber during a given disinfection cycle. A second benefit of a heating cycle is that it reduces the moisture content in the interior cavity of the disinfection device to allow the one or more objects therein to dry. A benefit of the specified temperature range is that it is sufficiently low to limit damage to the one or more objects in the disinfection device, particularly objects which are intended for single use since they are typically made of materials which are not intended to be exposed to harsh elements, such as high temperatures. It is noted that the microdroplet generation cycle utilizes a very small amount of water (˜1 mL for some cycles) and, thus, the moisture content is not high after a disinfection cycle is completed. Because of that, the low specified temperature during the heating cycle is sufficient to dry the one or more objects placed in the disinfection device. During the heating cycle, one or more fans operate to elevate and maintain the temperature in the interior cavity of the disinfection device in a relatively uniform manner.

In some cases, the time frame of the disinfection protocol involving blocks 90 and 92 and optionally blocks 94 and 96 may generally be between approximately 20 minutes and approximately 45 minutes, and more preferably between approximately 20 and approximately 35 minutes. Several protocols were tested with varying reductions of viral loads and which are detailed in the examples and tables below. An example cycle time for creating a mixture of plasma and microdroplets denoted in block 90 may be between 5 minutes and approximately 15 minutes. An example cycle time for generating plasma without droplet formation denoted in block 92 may be between approximately 5 minutes and approximately 15 minutes. An example cycle time for the heating cycle denoted in block 96, if employed, may be between approximately 10 minutes and approximately 20 minutes. An example cycle time for the dwell time cycle denoted in block 94, if employed, may be between approximately 10 minutes and approximately 20 minutes.

Turning to FIG. 7, a flow chart is illustrated showing program instructions for affecting operation of the disinfection devices disclosed herein. As shown in block 80, the disinfection devices disclosed herein include program instructions for receiving a signal to conduct a disinfection process in the disinfection device. The signal may generally come from the user interface of the disinfection device upon a user activating an input control to activate a disinfection cycle. In some cases, the disinfection devices disclosed herein may further include program instructions to perform a pre-operational check of the disinfection device (not shown in FIG. 7) before activating any of its components to conduct a disinfection cycle. The program instructions may generally include sending and receiving various signals to sensors and components in the disinfection device to confirm their operation as discussed in detail with respect to block 86 of FIG. 6. As shown in blocks 102, 104, and 106 of FIG. 7, the disinfection devices disclosed herein may include program instructions for activating a fan, a cold plasma generator and a nebulizer of the disinfection device. The program instructions for such actions may generally include sending signals to the respective components for their activation. It is note that the program instructions for blocks 102, 104 and 106 may be activated in any sequence or any two or more of the program instructions may be activated at the same time.

As shown in block 110, the disinfection devices disclosed herein may include program instructions for terminating operation of the nebulizer after activating each of the components noted in blocks 102, 104 and 106, particularly after a set amount of time. In some cases, the disinfection device disclosed herein may include program instructions for a dwell time cycle after a mixture of plasma species and droplets are created as described in reference to blocks 90 and 94 of FIG. 6. In such cases, the program instructions are for terminating operation of its cold plasma generator at the same time, prior to or subsequent to terminating operation of the nebulizer as is denoted by block 108 and its connecting line from block 106 and its doubled arrowed line to block 110. As denoted in block 112, the disinfection devices disclosed herein may include program instructions for reactivating the cold plasma generator subsequent to a predetermined amount of dwell time. Regardless of whether a disinfection device include program instructions for such a dwell time, the disinfection devices disclosed herein may include program instructions for generating plasma without droplet formation for a predetermined amount of time as described in reference to block 92 in FIG. 6 and denoted in block 114 of FIG. 7.

In addition, the disinfection devices disclosed herein include program instructions for terminating operation of the cold plasma generator after the time as elapsed as denoted in block 116 of FIG. 7. In this manner, regardless of whether a disinfection device include program instructions for the aforementioned dwell time, the disinfection devices disclosed herein include program instructions for terminating operation of the cold plasma generator after a set time period subsequent to terminating operation of the nebulizer. In some cases, a disinfection device may have program instructions for maintaining the operation of at least one fan in the disinfection device for a dwell time after such a termination of the cold plasma generator. In any case, a disinfection device may optionally have program instructions for activating heating element of the disinfection device after terminating the cold plasma generator as denoted in block 118 of FIG. 7.

### EXAMPLES

Diameters of the microdroplets were measured using a laser diffraction droplet size analyzer (Malvern Instruments, USA). The analyzer operated at 10 kHz and averaged an output of 10,000 measurements per second using a 5 mW He—Ne laser, 10 mm diameter, with a Fourier lens and a detector array to capture the diffracted light. Based on testing, the average particle size generated under this system was 5 microns, which shows significant improvements over particles at 50 microns in size because of increased surface area.

A disinfection chamber having a configuration similar to disinfection device 10 in FIG. 2 was used to disinfection PPE, particularly disposable face masks. Airflow from the plasma chamber fan was set to 3.2 m/sec of flow velocity coming out of each of the three 1″ diameter holes on the bottom. That is 0.293 cubic meters of air mixed with plasma treated droplets per minute. The disinfection chamber is 53 liters or 0.053 cubic meters. At the set fan speed the full disinfection chamber volume is changed 5.5 times per minute or 55 times in 10 minutes, allowing for thorough mixing of plasma treated droplets with the PPE.

To test the validity of the system, petri dishes with E. coli were placed on shelves within the chamber. Ozone measurements were taken via electric ozone sensors as well as paper strips, and additional tests, using paper strips, tested hydrogen peroxide, NO₃, and NO₂ within the fluid droplets.

Our samples showed that using the protocol of 10 minutes of plasma, 5 minutes of nebulization of fluid, followed by 10 minutes of 50° C. air recirculation we reached >6-log reduction of the bacteria on all materials and surfaces and >3-log reduction of virus.

TABLE 1
Results

| | E. Coli | Listeria | Salmonella | B. Cereus |
|---|---|---|---|---|
| 25 SLPM | 4-log | 4-log | 4-log | 4-log |
| 50 SLPM | 8-log | 8-log | 8-log | 8-log |
| 75 SLPM | 8-log | 8-log | 8-log | 8-log |

Accordingly, using a fan that generates approximately 0.3 m3 (300 SLPM) airflow per minute, we were able to maximize the log reduction of the bacterial loads. This optimizes the flow of air over the dielectric barrier discharge and the intermixing with the nebulized microdroplets to create the optimized RONS for antimicrobial activity.

Using the five-micron-sized microdroplets resulted in a >8-log reduction of E. coli, Listeria, Salmonella, and B. cereus on various surfaces, as well as B. cereus spore inactivation. On the fabric surface of a material similar to N95 respirator masks, a >3-log reduction of MS2 phage (nonenveloped virus) was observed. We utilized this virus as a reasonable viral sample that is difficult to kill than enveloped viruses. We also tested Phi6, which serves as a surrogate for COVID-like viruses. The FDA recommends that the viricidal activity is a >3-log reduction, and our studies showed that these levels were reached by the final protocol.

At 25 SLPM we found a 2-log reduction for all viruses, and at 50 and 75 SLPM we found a 3-log reduction for all viruses.

Interestingly, we also tested another protocol, which was 5 minutes of plasma nebulizer activation followed by 5 minutes of dwell time and finally 10 minutes of dry cycle at 50° C. This resulted in a 2-log reduction on plates, and a 1-log reduction on PPE. Accordingly, the time for plasma generation is important to ensure sufficient concentrations of reactive species within the cleaning chamber. We also tested 15 minutes of plasma, 5 minutes of nebulizer, and 10 minutes of nebulizer, and this resulted in the same kill rates as the 10 minutes of plasma and 5 minutes of nebulizer. The goal is to have the lowest total cycle time while reaching the necessary kill rate.

We also tested the impacts of heat on the product with regard to time and chemistry. While temperatures above 40° C. were sufficient to destroy the radical chemistries, the drying and damage results varied widely. The following were observed with regard to temperature and time for the heat cycle. Using a 10-minute heat cycle, heat at 40° C. resulted in wet PPE, 45° C. also resulted in wet materials. At 50° C. the materials were dry, at 55° C., 60° C., and 75° C., the material was also dry in 10 minutes. However, we noticed some deformation of the materials at temperatures of 75° C., and the temperatures of 55C° and 60° C. were not necessary to improve the drying or the destruction of the radical chemistries. Accordingly, 50° C. was advantageous to meet all of the goals of destroying reactive species and drying the materials efficiently and safely.

We also tested a longer heating cycle of 20 minutes. At 20 minutes, the 40° C. remained slightly damp, while at 45° C. it was dry. However, the longer time made the entire protocol take longer than using 50° C. Accordingly, 50° C. ±2° C. appears to be an optimized value.

### Use of Di Water, RO-DI Water, and Tap Water

We tested whether the fluid source would impact the kill rates. Here the three different samples yielded surprisingly different results. While tap water yielded the 8-log reductions, use of DI and RO-DI water in identical run cycles reduced yield to 5-log in each case. Surprisingly, the inclusion of small amounts of metals, dissolved salts, and such improved the kill rate on the tested plates.

The materials tested and expected for common use in the devices includes the following listed in Table 2 below.

TABLE 2

| What is being measured | On what surface | Log Reduction |
|---|---|---|
| E. coli inactivation | Agarose gel | 4 |
| E. coli inactivation | Mask fabrics | 3 |
| E. coli inactivation | Face Shields Plastics | 5 |
| Pseudomonas syringae inactivation | Agarose gel | 4 |
| Pseudomonas syringae inactivation | Mask fabrics | 3 |
| Pseudomonas syringae inactivation | Face Shields Plastics | 5 |
| Bacillus subtilis inactivation | Agarose gel | 3 |
| Bacillus subtilis inactivation | Mask fabrics | 2 |
| Bacillus subtilis inactivation | Face Shields Plastics | 4 |
| Aspergillus niger inactivation | Agarose gel | 3 |
| Aspergillus niger inactivation | Mask fabrics | 2 |
| Aspergillus niger inactivation | Face Shields Plastics | 4 |
| MS2 phage (virus) inactivation | Mask fabrics | 3 |
| MS2 phage (virus) inactivation | Face Shields Plastics | 4 |
| Phi6 phage (virus) inactivation | Mask fabrics | 3 |
| Phi6 phage (virus) inactivation | Face Shields Plastics | 4 |
| PhiX174 phage (virus) inactivation | Mask fabrics | 3 |
| PhiX174 phage (virus) inactivation | Face Shields Plastics | 4 |
| PM2 phage (virus) inactivation | Mask fabrics | 3 |
| PM2 phage (virus) inactivation | Face Shields Plastics | 4 |
| Human influenza A virus H1N1 inactivation | Mask fabrics | 3 |
| 4Human influenza A virus H1N1 inactivation | Face Shields Plastics | 4 |
| Newcastle disease virus inactivation | Mask fabrics | 3 |
| Newcastle disease virus inactivation | Face Shields Plastics | 4 |
| SARS-CoV-2 inactivation | Mask fabrics | 3 |
| SARS-CoV-2 inactivation | Face Shields Plastics | 4 |

Accordingly, from all our studies we tested the following parameters, and identified the optimized protocol to meet the necessary log reductions, having a short cleaning cycle, ensuring dry products, and reducing damage to the PPE. Study parameters are listed below.

TABLE 3

| | | | Highest log inactivation | |
|---|---|---|---|---|
| Parameter | Range studied | | achieved at | |
| Plasma on time | 3-15 | minutes | 10 | minutes |
| Plasma fan speed | 0.1-0.5 | m3/min | 0.3 | m3/min |
| Nebulizer on time | 1-15 | minutes | 5 | minutes |
| Nebulization flow rate | 0.05-0.5 | mL/min | 0.2 | mL/min |
| 50° C. heat + fan time | 5-20 | minutes | 10 | minutes |

Therefore, an optimized protocol uses a 10-minute plasma cycle with a fan speed of 0.3 m3/min. The nebulizer is running for the first 5 minutes of the plasma cycle at 0.2 mL/min of fluid, and finally a 10-minute dry cycle. This protocol optimized results by increasing kill rate and reducing run time and wear and tear on the PPE.

It will be appreciated to those skilled in the art having the benefit of this disclosure that this invention is believed to provide disinfection devices that do not cause damage to items being disinfected, sufficiently access all surfaces of items being disinfected in a relatively short amount of time and are economical. Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims. The term “approximately” as used herein refers to variations of up to +/−5% of the stated number.

What is claimed is:

1. A disinfection device, comprising:
an enclosure comprising an interior cavity;
a closable loading port for providing access to the interior cavity;
a chamber distinct from the interior cavity and having an air inlet and air outlet respectively coupled to an outlet and an inlet of the interior cavity for providing airflow between the chamber and the interior cavity;
a cold plasma generator disposed in the chamber;
a fan arranged within or in proximity to the outlet of the interior chamber such that air from the interior cavity is forced through the air inlet of the chamber and routed to the cold plasma generator; and
a nebulizer, wherein the cold plasma generator, the fan, and the nebulizer are arranged in the disinfection device such that:
plasma species generated by the cold plasma generator are routed to an area to which droplets formed by the nebulizer are discharged; and
the interior cavity is exposed to a mixture of the droplets and the plasma species.

2. The disinfection device of claim 1, wherein the nebulizer is arranged in the chamber downstream from the cold plasma generator.

3. The disinfection device of claim 1, wherein the nebulizer is arranged in the interior cavity.

4. The disinfection device of claim 3, wherein the nebulizer is arranged within 12 inches of the air outlet of the chamber and such that a nozzle of the nebulizer discharges droplets toward an area in the interior cavity adjacent the air outlet of the chamber.

5. The disinfection device of claim 1, further comprising an additional chamber distinct from the interior cavity, wherein the nebulizer is arranged in the additional chamber, and wherein the additional chamber comprises a fluid outlet for passing droplets formed by the nebulizer into the interior cavity.

6. The disinfection device of claim 5, wherein the air outlet of the chamber in which the cold plasma generator is arranged and the fluid outlet of the additional chamber are configured in the disinfection device to direct their discharge to a common area in the interior cavity which is within approximately 12 inches of each of the air outlet and the fluid outlet.

7. The disinfection device of claim 1, wherein the nebulizer is configured and arranged in the disinfection device such that droplets having a diameter of 10 microns or greater are discharged into the interior cavity.

8. The disinfection device of claim 1, further comprising an electronic control system comprising a processor and a storage medium comprising program instructions executable by the processor for:
activating the fan;
activating the cold plasma generator;
activating the nebulizer;
terminating operation of the nebulizer; and
terminating operation of the cold plasma generator after a set time period subsequent to terminating operation of the nebulizer.

9. A disinfection device, comprising:
an enclosure comprising an interior cavity;
a closable loading port for providing access to the interior cavity;
a cold plasma generator, wherein the disinfection device is configured such that plasma species generated by the cold plasma generator are discharged in the interior cavity;
a nebulizer, wherein the disinfection device is configured such that droplets generated by the nebulizer are discharged in the interior cavity;
a fan arranged in the disinfection device to move air, the plasma species and the droplets in the disinfection device; and
an electronic control system comprising a processor and a storage medium comprising program instructions executable by the processor for:
activating the fan;
activating the cold plasma generator;
activating the nebulizer;
terminating operation of the nebulizer; and
terminating operation of the cold plasma generator after a set time period subsequent to terminating operation of the nebulizer.

10. The disinfection device of claim 9, wherein the set time period is between approximately 5 minutes and approximately 15 minutes.

11. The disinfection device of claim 9, wherein the program instructions for terminating the operation of the nebulizer comprises terminating the operation of the nebulizer while maintaining operation of the fan and the cold plasma generator.

12. The disinfection device of claim 9, wherein the storage medium further comprises program instructions executable by the processor for:
terminating operation of the cold plasma generator prior to or at the same time as the step of terminating operation of the nebulizer;
reactivating the cold plasma generator, wherein the step of terminating operation of the cold plasma generator after a set time period subsequent to terminating operation of the nebulizer comprises terminating operation of the reactivated cold plasma generator; and
maintaining operation of the fan between the steps of terminating operation and reactivating the cold plasma generator.

13. The disinfection device of claim 9, further comprising a heating element arranged in the disinfection device to heat the interior cavity, wherein the storage medium further comprises program instructions executable by the processor for activating the heating element at the same time or subsequent to the step of terminating operation of the cold plasma generator.

14. The disinfection device of claim 9, wherein the program instructions for activating the fan comprise program instructions for activating the fan to operate at a flow rate of at least 0.3 m³ per minute.

15. A disinfection device, comprising:
an enclosure comprising an interior cavity;
a closable loading port for providing access to the interior cavity;
a chamber distinct from the interior cavity and having an air inlet and air outlet respectively coupled to an outlet and an inlet of the interior cavity for providing airflow between the chamber and the interior cavity;
a cold plasma generator disposed in the chamber;
a fan arranged within or in proximity to the outlet of the interior chamber such that air from the interior cavity is forced through the air inlet of the chamber and routed to the cold plasma generator and that plasma species generated by the cold plasma generator are discharged into the interior cavity;
a nebulizer arranged in the disinfection device such that droplets formed by the nebulizer are discharged in the interior cavity; and
a heating element to heat the interior cavity.

16. The disinfection device of claim 15, wherein the heating element and the disinfection device are configured to raise and maintain a temperature in the interior cavity to be between approximately 40° C. and approximately 60° C. for a predetermined amount of time.

17. The disinfection device of claim 16, further comprising an electronic control system comprising a processor and a storage medium, wherein the storage medium comprises program instructions executable by the processor for:
activating the cold plasma generator;
terminating operation of the cold plasma generator; and
activating the heating element at the same time or subsequent to the step of terminating operation of the cold plasma generator.

18. The disinfection device of claim 17, wherein the storage medium further comprises program instructions executable by the processor for:
activating the nebulizer; and
terminating operation of the nebulizer prior to the step of terminating the cold plasma generator.

19. The disinfection device of claim 15, wherein the nebulizer is configured to generate microdroplets having a mean diameter of approximately 5 μm or less.

20. The disinfection device of claim 15, wherein the interior cavity comprises a volume between approximately 0.1 liters and approximately 100 liters.
